(19) United States
(12) Patent Application Publication
(10) Pub. No.: US 2016/0067275 A1
(43) Pub. Date: Mar. 10, 2016

(54) CO-CROSSLINKED PHOSPHATED NATIVE AND/OR FUNCTIONALIZED POLYSACCHARIDE-BASED HYDOGEL

(71) Applicant: AYAWANE, Lyon (FR)

(72) Inventor: Latifa DAHRI CORREIA, l'Isle d'Abeau (FR)

(73) Assignee: AYAWANE, Lyon (FR)

(21) Appl. No.: 14/786,980

(22) PCT Filed: Apr. 23, 2014
(86) PCT No.: PCT/FR2014/050984

§ 371 Date: Oct. 25, 2015

(30) Foreign Application Priority Data
Apr. 24, 2013 (FR) 1353719

(51) Int. Cl. A61K 31/738

(52) U.S. Cl. CPC A61K 31/738

(57) ABSTRACT

Phosphorylated hydrogels obtained by co-cross-linking hyaluronic acid with dextran, a process for preparing same, and a use of the hydrogel for the encapsulation and extended release of active principles as well as cells for use in regenerative human and veterinary medicine.

### CROSS-REFERENCE TO RELATED APPLICATIONS

The present application is a National Stage Application of PCT International Application No. PCT/FR2014/050984 (filed on Apr. 23, 2014), under 35 U.S.C. §371, which claims priority to French Patent Application No. 1353719 (filed on Apr. 24, 2013), which are each hereby incorporated by reference in their respective entireties.

### TECHNICAL FIELD

The invention relates to the field of hydrogels based on hyaluronic acid. In particular, it relates to phosphorylated hydrogels obtained by co-cross-linking hyaluronic acid with dextran, as well as the process for preparing same. The invention also relates to the use of said hydrogel for the encapsulation and extended release of active principles as well as cells for use in regenerative human and veterinary medicine.

### BACKGROUND

Hyaluronan or hyaluronic acid (CAS no. 9004-61-9, also abbreviated HA in this document) belongs to the glycosaminoglycan family. It is a disaccharide polymer, more specifically a linear non-sulfated glycosaminoglycan comprised of repetitive units of glucuronic and N-acetyl-D-glucosamine D-acid bound together by alternating beta-1,4 and beta-1,3 glycosidic bonds (see the publication of Tammi et al. “Hyaluronan metabolism in skin” published in the journal Progress in Histochemistry & Cytochemistry, 29 (2): 1-81, 1994)).

Hyaluronic acid is found in many mammal tissues, and more specifically in the extracellular tissue matrix. Owing to its capacity to bind water molecules, its main role is tissue hydration. If its molecular mass exceeds around 2 MDa and its mass fraction around 0.1%, it forms a hydrogel; according to its molecular mass and its mass fraction, it is present in this form in certain tissues, for example in the joints, and in particular in the synovial fluid. It also performs other roles, for example in the regulation of biological phenomena such as cell migration, tissue differentiation, angiogenesis and immune cell regulation. Thus, the prior art attributes fairly different functions to this molecule on the basis of its molecular mass.

Hyaluronic acid is broken down in the body by a group of enzymes called hyaluronidases (see the article “The magic glue hyaluronan and its eraser hyaluronidase: A biological overview” by K. S. Girish and K. Kemparaju, published in Life Sciences 80 (2007), p. 1921-1943); other enzymes are involved in this process.

Hydrogels based on hyaluronic acid are known and used in numerous medical applications, in particular in an injectable form in cutaneous reconstruction, cosmetic surgery and dermatology, or in orthopedics for cartilaginous and osteoarticular reconstruction (for example, for viscosupplementation of the knee). They are also used as a cell culture medium for research and cell therapy, or in chemotherapy.

According to the mode of preparation and the uses thereof, injectable hydrogels based on hyaluronic acid may be classified into three distinct categories:
“conductive” hydrogels, comprised of hyaluronic acid (HA) cross-linked alone or in combination with another polymer, used to fill wrinkles in cosmetic surgery or in viscosupplementation (restoration of homeostasis);
“inductive” hydrogels, comprised of cross-linked hyaluronic acid, containing growth factors and/or other so-called bioactive molecules for controlling their in situ delivery;
hydrogels for cell implantation consisting of multiple components: polymers, proteins and cells.

For use in human or veterinary medicine, a hydrogel must satisfy numerous criteria (cf. M. N. Collins and C. Birkinshaw, “Hyaluronic acid base scaffolds for tissue engineering—A review”, Carbohydrate Polymers 92 (2013), 1262-1279; 2013; G. D. Prestwich, “Hyaluronic acid-based clinical biomaterials derived for cell and molecule delivery in regenerative medicine”, J. Controlled Release 155 (2011), 193-199). For example, the hydrogel must be biodegradable, biocompatible and resorbable; and it must be easy to use under physiological conditions.

Normally, the term “biocompatibility” refers to the capacity of a non-living material called upon to interact with biological systems to satisfy a specific function with an appropriate response to the host.

Collagen, a glycoprotein family, has long been used as a resorbable, injectable filling product. However, collagen has certain disadvantages, which are: the risk of appearance of allergic reactions and rapid degradation in vivo.

These are the reasons for which it is replaced by hyaluronic acid salts, which do not present any allergic reaction and are biocompatible regardless of their origin and mode of preparation (animal, biotechnology).

Today, hydrogels of hyaluronic acid, in particular salts and/or derivatives thereof, have been involved to a significant extent in the field of cosmetics, in particular for filling wrinkles and cutaneous defects.

Hyaluronic acids and salts thereof may be used alone for the production of hydrogel, but may also be associated with other polymers (collagen, gelatin, chitosan, chondroitin sulfate, and so on) to promote a synergy of the different components, and more reliably mimic the extracellular matrix.

Hyaluronic acid may be chemically modified owing to carboxylic, hydroxyl and acetamide functions, present along the chain. These groups may be used, on the one hand, for cross-linking, and, on the other hand, for functionalization in order to provide new biological properties or improve intrinsic biological and/or physicochemical properties. Cross-linking involves the creation of bonds between two polysaccharide chains and/or between two segments of the same chain. It results in an increase in viscosity and/or molecular mass, and drives the modification of other physical and physicochemical properties.

Thus, there are numerous possibilities for preparing hydrogels from hyaluronic acid.

The document FR 2 918 377 describes a cohesive co-cross-linked gel including at least one first strongly cross-linked polysaccharide gel, and at least one second weakly cross-linked polysaccharide gel, the first gel being bound by covalent bonds to the second gel. The strongly cross-linked gel and the weakly cross-linked gel are preferably based on sodium hyaluronate; the cross-linking agent is 1,4-butanediol diglycidyl ether (BDDE).

The document U.S. Pat. No. 6,586,493 describes a hydrogel obtained from a mixture including a partially unsaturated polysaccharide, substituted by a cross-linking group, and a non-angiogenic hyaluronic acid. The cross-linking of the mixture is performed by adding a free radical polymerization initiator, in particular an acetophenone (photo-initiators). This process is very disadvantageous because numerous degradation products resulting from free radical polymerization may be trapped in the hydrogel once formed.

This invention is intended to present new hydrogels based on polysaccharides, having good resistance to increased degradation and an absence of cytotoxicity, which are injectable, and biocompatible for use in cosmetic surgery, dermatology, ophthalmology, orthopedics for cartilaginous and osteoarticular reconstruction and in regenerative human and veterinary medicine.

### SUMMARY

This invention relates to a co-cross-linked phosphorylated polysaccharide hydrogel, based on (i) dextran and (ii) hyaluronic acid and/or one of its salts, optionally functionalized, in which hydrogel said dextran and said hyaluronic acid (and/or its salt, optionally functionalized) are bound together by phosphodiester and/or polyphosphodiester covalent bonds.

Advantageously, the hyaluronic acid, or its salt, is a phosphoryl hyaluronic acid (and/or its salt) functionalized by sodium trimetaphosphate.

In a particular embodiment of the invention, the phosphate concentration of the hyaluronic acid (and/or its salt) functionalized by sodium trimetaphosphate is between 0.01 and 4 mEq/g, more preferentially between 0.1 and 2 mEq/g.

In another embodiment of the invention, the phosphate concentration of the hydrogel is between 0.1 and 3 mEq/g, more preferentially between 0.1 and 2 mEq/g.

This invention also relates to a process for preparing a co-cross-linked phosphorylated polysaccharide hydrogel, based on dextran and hyaluronic acid and/or one of its salts, optionally functionalized, in which process:

a) a dextran solution is provided;

b) at least one hydroxyl group of the dextran is activated by adding an alkaline hydroxide solution, for example sodium hydroxide, to said dextran solution to obtain an activated dextran solution;

c) sodium trimetaphosphate is added to said activated dextran solution;

d) hyaluronic acid and/or one of its salts, optionally functionalized, is added to the solution obtained in step c).

Advantageously, steps c) and d) of the process are performed concomitantly, the sodium trimetaphosphate and hyaluronic acid and/or one of its salts, optionally functionalized, being added concomitantly to the solution obtained in step b) of said process.

Advantageously, steps a) through d) of said process are performed at a temperature of between 18 and 25° C.

In a particular embodiment of the process, the concentration of alkaline hydroxide added in step b) is between 0.1 M and 1 M, preferably between 0.1 M and 0.5 M.

In another particular embodiment of the process, the hyaluronic acid and/or one of its salts added in step d) of the process is a hyaluronic acid functionalized by sodium trimetaphosphate.

Advantageously, the hyaluronic acid and/or one of its salts, optionally functionalized, is added in the form of a powder.

This invention also relates to a hydrogel according to the invention, and a hydrogel capable of being obtained by a process according to the invention, for preparing a drug, intended to be implanted alone or associated with cells (optionally stem cells) for cell therapy, or intended to be used in wet form for topical application, or intended to deliver active molecules and active principles, and more specifically growth factors, preferably growth factors selected from: fibroblast growth factors (FGFs), platelet derived growth factors (PDGFs), transformation growth factors (TGFs), vascular endothelium growth factors (vEGF), osteoinductive growth factors (BMPs); or intended to be used in regenerative medicine, and more specifically in human and/or veterinary regenerative medicine.

A final objective of the invention is the use of a hydrogel according to the invention for osteoconductivity and/or osteoinductivity, i.e. for bone regeneration.

### DRAWINGS

FIGS. 1 to 8 illustrate certain aspects of the invention.

FIG. 1 is an infrared spectrum (IR) showing the change in the infrared profile of a reaction mixture of the process according to the invention (example 1) over 24 hours starting from the concomitant addition of hyaluronic acid and sodium trimetaphosphate TMP (curve a: 22 minutes after addition; curve b: 32 minutes after addition; curve c: 42 minutes after addition; curve d: 52 minutes after addition; curve e: 120 minutes after addition; curve f: gel obtained after 24 h at 25° C.). The x-axis shows the number of waves expressed in cm-1; the y-axis shows the transmittance (in %, without dimension).

FIG. 2 is a graph showing the influence of the alkaline hydroxide concentration and the sodium trimetaphosphate concentration on the cross-linking of dextran.

FIG. 3 is a 31P NMR spectrum of co-cross-linked hydrogel prepared according to the invention after 24 hours.

FIG. 4 shows two 31 P NMR spectra of sodium trimetaphosphate at 25° C. in the presence of NaOH at 3M.

FIG. 5 is a 31P NMR spectrum making it possible to determine the quantity of phosphate of a sample of gel dehydrated by hydrolysis in a strong acid medium (HCl 4N).

FIG. 6 is a graph showing the impact of the hyaluronic acids functionalized (HH) by sodium trimetaphosphate on the proliferation of human dermal fibroblasts as a function of their concentration and their degree of functionalization (read by spectrophotometry at 450 nm).

FIG. 7 is a graph comparing the mass percentage of fibroblast growth factors not absorbed by hydrogels according to the invention after their contact with hydrogels for 24 hours in an NaCl solution.

FIG. 8 is a graph showing the impact of the hydrogels according to the invention on cell proliferation, and in particular on the proliferation of stem cells called “circulating endothelial progenitors.”

### DESCRIPTION

In the disclosure of the present invention, the term “hyaluronic acid” includes hyaluronic acid salts.

The term “phosphorylated hydrogel” refers to a hydrogel, the chemical structure of which includes at least one phosphodiester and/or polyphosphodiester covalent bond.

The term “co-cross-linking agent” refers to an agent enabling the formation of covalent bonds between two different polysaccharides, more specifically between a hyaluronic acid, optionally functionalized, or one of its salts, and dextran; the co-cross-linking agent being chosen from a cross-linking agent itself known for performing the cross-linking of these same polysaccharides. Thus, in the context of this invention, the terms “co-cross-linking agent” and “cross-linking agent” refer to the same chemical agent (i.e. sodium trimetaphosphate).

The term “co-cross-linked polysaccharide hydrogel” refers to a hydrogel including at least two different polysaccharides bound together by covalent bonds.

The term “co-cross-linked phosphorylated polysaccharide hydrogel” refers to a hydrogel including at least two different polysaccharides bound together by phosphodiester and/or polyphosphodiester covalent bonds as shown below (R1 and R2 respectively show a first and a second substituted polysaccharide).

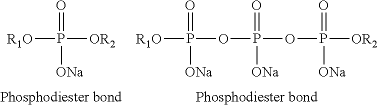

The term “functionalized hyaluronic acid” (also abbreviated HH in this document) refers to a hyaluronic acid (or one of its salts) substituted (at least partially) by various organic groups.

In the following detailed description of the invention, the reaction mechanisms mentioned are merely hypotheses and heuristic models that do not limit the scope of the invention.

In the context of this invention, the applicant has demonstrated that a hyaluronic acid, optionally functionalized, or one of its salts, in a mixture with an activated dextran, using sodium trimetaphosphate as a cross-linking agent, enables a co-cross-linked phosphorylated hydrogel having improved biological properties to be obtained. The applicant has demonstrated that the objectives of the invention are achieved by obtaining a co-cross-linked phosphorylated polysaccharide hydrogel obtained by a specific and optimized process described in greater detail below.

According to the invention, the hyaluronic acids may optionally be used in the form of a physiologically acceptable salt, and may be functionalized.

According to a first aspect, this invention relates to a process for preparing a co-cross-linked phosphorylated polysaccharide hydrogel, said process including the following steps:

a) A dextran solution is provided;

b) At least one hydroxyl group of the dextran is activated by adding an alkaline hydroxide solution (for example, sodium hydroxide NaOH) to said dextran solution; this step leads to the transformation of said at least one hydroxyl group into an alcoholate group, and the dextran thus transformed is called “activated dextran”, according to the reaction:

DexOH+NaOH→DexONa

c) Sodium trimetaphosphate is added to said activated dextran solution. This step leads to the cross-linking of the activated dextran by a nucleophilic attack of the oxygen of the dextran alcoholate group on a phosphorus of the trimethylphosphate, as indicated in the reaction scheme 1.1 below. A plurality of reactions may occur during the cross-linking according to the reaction schemes below (the arrow coming from the DexONa symbolizes the nucleophilic attack of the oxygen of the dextran alcoholate on the phosphorus of the trimetaphosphate).

### Reaction Schemes 1: Possible Cross-Linking Reactions of Activated Dextran

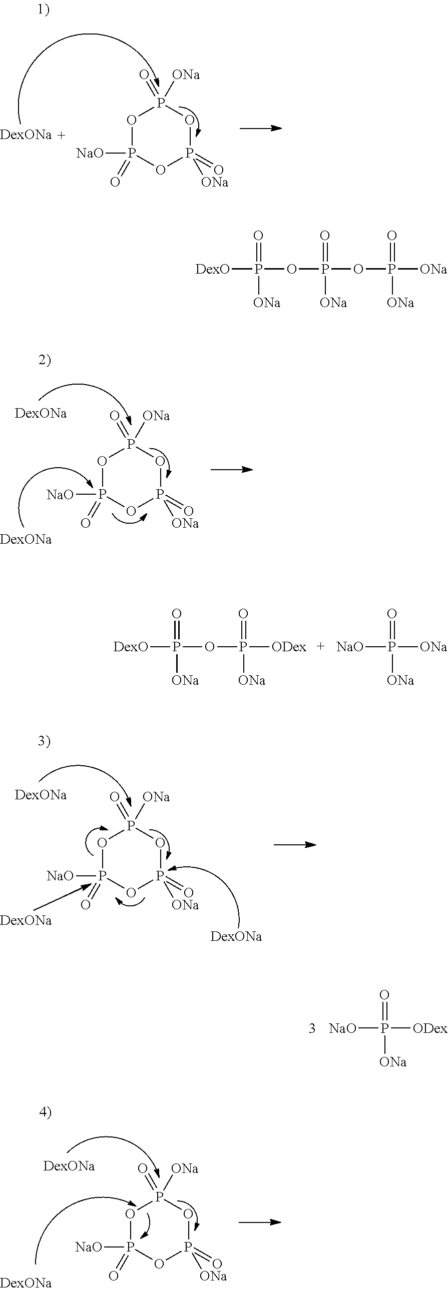

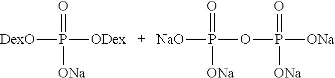

d) Hyaluronic acid (HA), optionally functionalized (HH), or one of its salts, is added to the solution obtained in step c); preferably, this addition is performed with powder and not with a hyaluronic acid solution (optionally functionalized, or one of its salts). During the addition of the hyaluronic acid (HA or HH) and during the formation of the hydrogel, the reaction medium is kept under agitation. The temperature during this step is advantageously between 18° C. and 25° C.

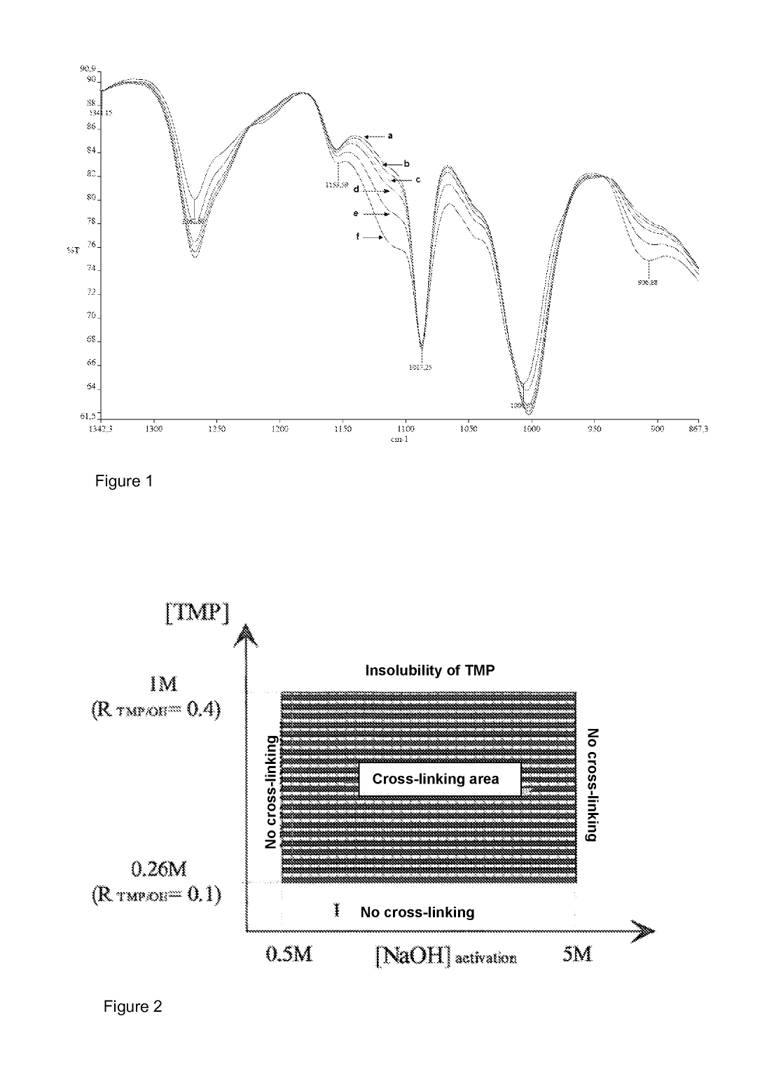

This step d) leads to the co-cross-linking of activated dextran with hyaluronic acid, optionally functionalized, or one of its salts, to form a co-cross-linked phosphorylated polysaccharide hydrogel, the mixture being kept under agitation until the hydrogel is obtained. The reaction time is typically between 10 and 40 minutes (preferably between 20 and 30 minutes) in order to form the hydrogel. Then, the fixed hydrogel is left at room temperature (typically 18-25° C.) in order for the reaction to be capable of ending. The analysis by Fourier-transform infrared spectroscopy (FT-IR) shows the presence of phosphoric esters on the polysaccharide chain (cf. FIG. 1 and explanations provided in the examples).

The phosphorylated hydrogel according to the invention therefore includes a first polysaccharide (dextran), and a second polysaccharide (hyaluronic acid, optionally functionalized, or one of its salts), said polysaccharides being bound together by phosphodiester and/or polyphosphodiester covalent bonds.

Sodium trimetaphosphate or STMP (CAS no. 7785-84-4), with the molecular formula Na3P3O9, is a nontoxic compound for humans (see, for example: K. Woo and P. A. Seib, Carbohydrate Polymers 1997 (33), 263-271), commonly used in the food industry. Sodium trimetaphosphate may undergo nucleophilic attacks thus enabling the STMP ring to open. A reaction mechanism describing the nucleophilic attack of a polysaccharide alcoholate on sodium trimetaphosphate has been proposed in the scientific literature (see the article “High-resolution nuclear magnetic resonance spectroscopy studies of polysaccharides cross-linked by sodium trimetaphosphate: a proposal for the reaction mechanism” by S. Lack et al. published in Carbohydrate Research 342 (2007) p. 943-953).

The use of sodium trimetaphosphate as a cross-linking and co-cross-linking agent is essential in the process according to the invention, because it makes it possible to obtain water-insoluble cross-linked phosphated polysaccharides. In addition, owing to the specific use of this chemical agent and the synergistic effect between the hyaluronic acid, optionally functionalized, and the dextran, the co-cross-linked phosphorylated polysaccharide hydrogel according to this invention has a better resistance to enzymatic degradation by hyaluronidases (enzymes responsible for degradation of hyaluronic acid in the body) than a simply cross-linked hyaluronic acid hydrogel (i.e. a hyaluronic acid hydrogel cross-linked to itself, without the addition of another polysaccharide). Finally, the hydrogel according to the invention has a very clear improvement with regard to cell proliferation.

The conditions for preparation of a co-cross-linked phosphorylated hydrogel are presented below.

First, a hydrogel based on cross-linked dextran, by means of sodium trimetaphosphate, is made. The cross-linking reaction of dextran occurs in two steps; the first step consists in the activation of the most reactive hydroxyl groups of dextran (i.e. the hydroxyl groups in C2 of dextran) in order to form a dextran alcoholate (also called activated dextran in this document), then in a nucleophilic attack of the dextran alcoholate on the sodium trimetaphosphate (cf. the reaction schemes above).

During the cross-linking reaction, certain secondary reactions are observed. These are in particular basic hydrolysis of the phosphated bridges formed, thus solubilizing the hydrogel (cf. reaction scheme no. 2 below). Another secondary reaction is the basic degradation of sodium trimetaphosphate into sodium tripolyphosphate (TPP) (cf. reaction scheme no. 3 below). These secondary reactions are not desirable in the context of this invention.

### Reaction Scheme No. 2: Example of Basic Hydrolysis of the Phosphate Bridges

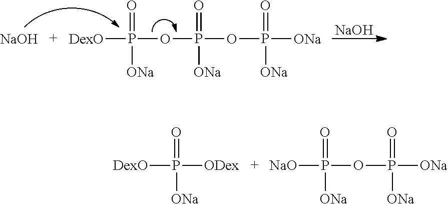

Reaction Scheme No. 3: Basic Degradation of the Sodium Trimetaphosphate into Tripolyphosphate (TPP)

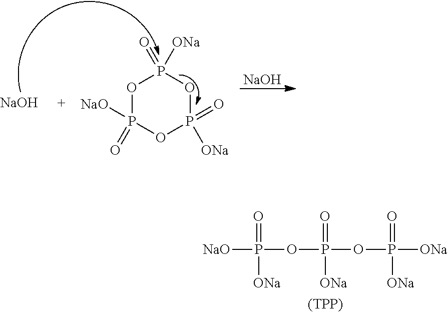

To limit these secondary reactions, the inventor has found an optimal range for the alkaline hydroxide and sodium trimetaphosphate STMP concentration (cf. FIG. 2: the alkaline hydroxide concentration indicated in FIG. 2 corresponds to the alkaline hydroxide concentration during the dextran activation phase; the STMP concentration indicated corresponds to the STMP concentration on the reaction medium (dextran+alkaline hydroxide+STMP)). The hydroxide concentration is preferably between 0.5 M and 5 M in order to activate the hydroxyl groups of the dextran. In a preferred embodiment, the hydroxide concentration during said activation step is between 0.5 M and 1 M. Similarly, the STNMP concentration in the reaction medium is preferably between 0.26 M and 1 M, which corresponds to a ratio [STMP]/[OH] of between 0.1 and 0.4. In fact, for a hydroxide concentration above 5 M, the basic hydrolysis of the phosphate brides is promoted to the detriment of the cross-linking of the dextran. Moreover, for an STMP concentration below 0.26, no cross-linking is observed, and for a concentration above 1 M, the STMP becomes insoluble in water.

Advantageously, the process according to the invention is performed at a temperature of between 18 and 25° C. In fact, even if an increase in the temperature of the reaction medium has the effect of reducing the gelling time of the solution, it accelerates the hydrolysis of the phosphate bridges formed. This is why it is more advantageous to perform the process at room temperature in order to limit the hydrolysis of the phosphate bridges formed. Below 18° C., the cross-linking reaction becomes very slow.

In the preparation process described, the different steps of the process are performed in a basic medium, the pH value of which is substantially the same. Each of the steps is performed in a basic medium at a pH capable of being between 8 and 14, preferably between 8 and 10.

In a very advantageous embodiment of the process according to the invention, the hyaluronic acid, optionally functionalized, or one of its salts, is added to the reaction medium at the same time as the sodium trimetaphosphate (and separately); steps c) and d) of the process according to the invention are thus combined. The applicant was able to observe that the best gelling results were obtained by this method. In fact, for questions of stability of the hyaluronic acid in a basic medium (or functionalized hyaluronic acid), it is advantageous to add it at the same time as the sodium trimetaphosphate. This addition of HA and/or HH is performed advantageously in the form of a powder, and the same applies to the sodium trimetaphosphate; this increases the cross-linking and decreases the magnitude of the secondary reactions with respect to an addition in solution. In every case, whether it is performed in the form of a powder (preferred) or in the form of a solution (less preferred), the simultaneous addition of HA and/or HH, on the one hand, and sodium trimetaphosphate, on the other hand, is advantageously performed in separate spaces, i.e. the addition of powders and/or solutions, which are separate, is performed at different locations of the reaction mixture.

Preferably, the hyaluronic acid (HA and/or HH) used in the process according to this invention has a molecular mass of between 10 and 5000 kDa, preferably between 10 kDa and 1200 kDa, and more preferably between 25 and 1200 kDa. This choice is motivated by the use of the hydrogel according to the invention including a hyaluronic acid, optionally functionalized, phosphorylated with molecular mass for its biological effects:
the hyaluronic acid of low molecular mass, i.e. below 50 kDa, preferably between 25 and 50 kDa, and more preferably between 10 and 50 kDa, is preferred for inducing an angiogenic effect;
the hyaluronic acid of molecular mass between 50 and 300 kDa is preferred for cell proliferation and migration;
the hyaluronic acid of molecular mass above 800 kDa and up to 5000 kDa is preferred for its effect in maintaining tissue hydration.

Preferably, the dextran used, in the process according to this invention, has a molecular mass of between 10 kDa and 2000 kDa, preferably between 40 kDa and 500 kDa, also for reasons associated with the use of the hydrogel according to the invention (given that this preference is less strong than that concerning the molecular mass of HA and HH).

In a particularly advantageous embodiment, the hyaluronic acid added in step d) of the process according to the inventions [this step d) being possibly performed at the same time as step c)] is a hyaluronic acid functionalized by sodium trimetaphosphate. The synthesis of this functionalized hyaluronic acid is performed in two steps. In a first so-called activation step, a sodium hydroxide solution is added in order to activate the hydroxyl groups of the hyaluronic acid. Preferably, the activation phase is performed at a basic pH below 8 (otherwise a new secondary reaction, namely the spontaneous opening of the cyclic trimetaphosphate molecule, may occur). Then, a so-called functionalization step is performed by adding sodium trimetaphosphate in the hyaluronic acid solution in alcoholate form.

After step d) of the process according to the invention, the functionalized, phosphorylated hyaluronic acid is purified and dried according to conventional techniques familiar to a person skilled in the art, and in particular the functionalized hyaluronic acid may be precipitated in ethanol (or acetone) in order to remove residual traces of co-cross-linking agent, then dialyzed against osmosis water until the pH and the conductometry of the dialysis water are close to those of the osmosis water (pH 5.6=and conductometry <30 ps/cm). The hyaluronic acid thus purified may be lyophilized.

The inventor has observed that the enzymatic degradation of the hydrogel according to the invention by the hyaluronase enzyme is faster than the degradation by the dextranase enzyme, and that the degradation with each of the two enzymes is slowed if the hydrogel is strongly cross-linked.

For the use of the hydrogel according to the invention as a medium for cell culture (in particular in research and cell therapy), an important aspect is the degree of functionalization of the hyaluronic acid by the phosphate. The improvement in cell proliferation is observed only starting at a phosphate content of 0.01 mEq/g. The content is preferentially at least 0.05 mEq/g, and even more preferentially at least 0.1 mEq/g (a unit “mEq/g” also called “milliequivalent/g” is equivalent to one millimole per gram of product).

The product according to the invention has numerous advantages, and in particular an excellent compromise of the following properties: physicochemical properties, biocompatibility, implantation, resistance to enzymatic degradation, absence of cytotoxicity. This result is obtained by optimizing the implementation of the co-cross-linking of hyaluronic acid with dextran in order to prepare a hydrogel based on co-cross-linked phosphorylated polysaccharides.

We indicate here, by way of example, several uses of the hydrogel according to the invention.

For the use of the hydrogel according to the invention in cell therapy, a hydrogel according to the invention is prepared and the appropriate cell nutrients (amino acids, etc.) and growth factors are incorporated. This product may be stored in lyophilized and/or frozen form. After rehydration of the lyophilized product, the hydrogel may be used in topical (for example as a dressing) or injectable form.

For the use of the hydrogel according to the invention as a system for the delivery of active principle, a hydrogel according to the invention is prepared in powder form, the hydrogel is hydrated (either with physiological serum or with a predetermined quantity of active principle(s), for example a growth factor and/or an anti-inflammatory and/or an antibiotic, or platelet-rich plasma in order to obtain a hydrogel that has the desired consistency (saturated gel)), and the product is deposited on the targeted site (for example, on a bone microfracture, a microtear or a tendon or muscle tear).

Other objectives, features and advantages of the invention will now be presented in the following examples, without in any way limiting the invention.

### Example 1

### Preparation of a Co-Cross-Linked Hydrogel Using a Hyaluronic Acid (HA) and Dextran (Process According to the Invention)

i) Activation of the Hydroxyl Groups of the Dextran with a 0.5 M NaOH Solution

0.9 grams of dextran T100 (CAS no. 9004-54-0; 100,000 g/mol) were placed in solution in 50 ml of water, then 5 ml of sodium hydroxide NaOH 0.5 M were added to the solution. The mixture was kept under agitation for 30 minutes at room temperature.

ii) 0.1 gram of hyaluronic acid (500K, mol wt. 400-600 kDa) ratio HA/HA+dextran=10%) and 1.02 grams of sodium trimetaphosphate (used as a cross-linking agent) were added simultaneously (in powder form, spatially separated) to the solution of step a).

The mixture was kept under agitation at room temperature until gelling, then the gel obtained was kept for 5 hours at 25° C.

iii) Washing, Purification and Drying of the Hydrogel

The hydrogel was ground on a stainless steel sieve and collected in an acetone solution. A plurality of washings in water were performed while monitoring the pH, until a pH<7 of the washing water was obtained; for each washing step performed, the hydrogel was filtered on a Büchner with a cellulose acetate filter. A final washing with acetone was performed, then the hydrogel was dried in the oven under vacuum at 60° C.

### iv) Chemical Characterization

The change in the co-cross-linking reaction by IR spectroscopy of the hyaluronic acid (500K, mol. Wt. 400-600 kDa) and dextran T100 is shown in FIG. 1. The experimental conditions were the following:

FT-IR “Spectrum One” spectrometer of Perkin Elmer Instruments, analyses performed by ATR (attenuated total reflection) probe.

The features of the main IR absorption bands are indicated in table 1 below.

TABLE 1
Characteristics of the main IR absorption bands

| Wave number (cm⁻¹) | Group | Vibration mode |
|---|---|---|
| 1260 | P═O (phosphonate) | Elongation |
| 1153 | secondary —OH | Elongation |
| 1087 | secondary —OH | Valence vibration (vC—O) |
| 1006 | primary —OH | Valence vibration (vC—O) |
| 906 | P—OR (phosphoric | Elongation |
| | ester) | |

During the co-cross-linking reaction, the monitoring of the reaction by infrared spectroscopy shows the nucleophilic attack of the alcoholates on the sodium trimetaphosphate, the characteristic bands of the primary alcohols (1006 cm⁻¹) decreasing at the expense of the characteristic bands of the secondary alcohols (1153 cm⁻¹). The IR absorption bands at 1260 cm⁻¹ and 906 cm⁻¹ show the formation of phosphoric and phosphonate ester (phosphodiester bond).

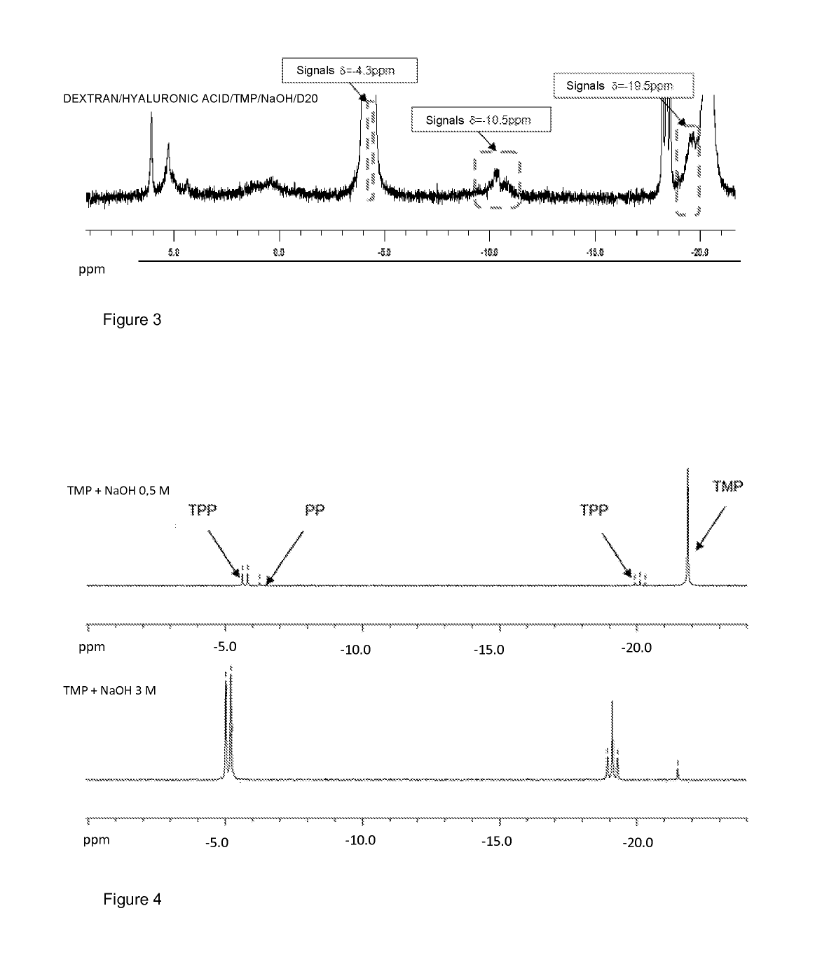

The ³¹P NMR spectrum (Bruker 250 MHz—NMR phosphorus analyses; mode: proton irradiation; frequency: 101 MHz) of the hydrogel collected was also analyzed (D₂O solvent) and is shown in FIG. 3. The presence of characteristic signals at displacements δ=−4.3 ppm; δ=−10.5 ppm and δ=−19.5 ppm show the formation of phosphoric esters.

### v) Determination of the Phosphate Content

A phosphate assay was performed using a hydrogel obtained according to the invention, dehydrated then hydrolyzed in an acid medium (HCl 4N) under the following conditions:

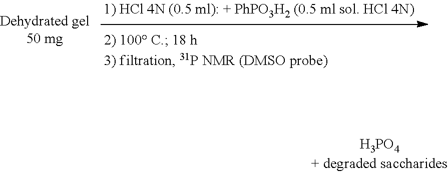

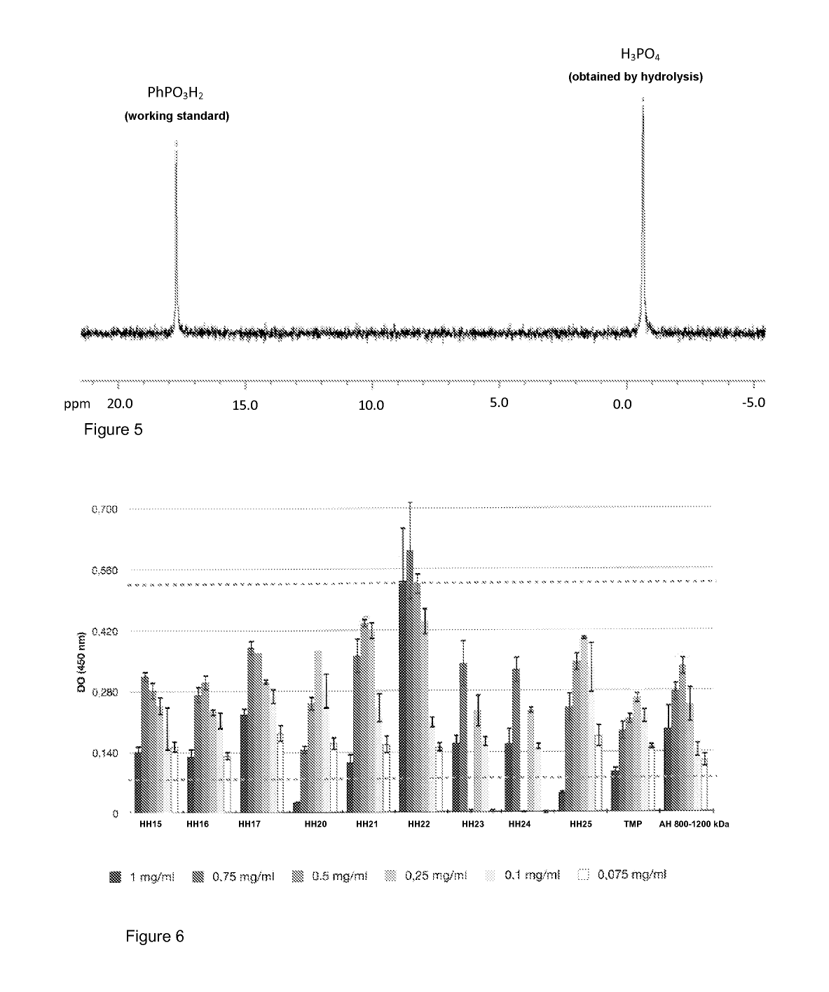

The phosphates and polyphosphates bound in the form of esters to the saccharide motifs are hydrolyzed in an acid medium. After treatment, they are indifferently transformed into monomer motifs of orthophosphoric acid. The 31 P NMR gives a signal, the characteristic displacement of which at −0.64 is characteristic of orthophosphoric acid. It is thus possible to determine the quantity of phosphate present in the hydrolysate. Phenylphosphonic acid PhPO3H2 is used as a standard (cf. FIG. 5).

Phosphate content: 1.06 mEq/g (1 mEq/g corresponds to 1 mmole of phosphoric acid generated after hydrolysis per gram of dehydrated gel analyzed).

### Example 2

### Preparation of a Hyaluronic Acid Functionalized (HH) by Sodium Trimetaphosphate (TMP): A Phosphorylated Hyaluronic Acid Obtained

The purpose of this example is to prepare a functionalized hyaluronic acid capable of being used in step d) of the process according to the invention.

0.20 grams of hyaluronic acid (Sigma Aldrich—CAS no. 9004-61-9—500K, 400-600 kDa) having a molecular mass distribution in the range of 400 kDa-600 kDa were placed in solution in 30 ml of water, then 0.3 ml of sodium hydroxide NaOH 0.1 M (CAS no.: 1310-73-2) were added to the solution. The mixture was kept under agitation for 30 minutes at room temperature.

0.09 grams of sodium trimetaphosphate (Sigma Aldrich CAS no. 7785-84-4) were solubilized in 10 ml of water, then added to the activated hyaluronic acid solution. The mixture was kept under agitation for 3 hours at room temperature.

The reaction medium was then neutralized by a 1M hydrochloric acid solution (CAS no. 7-647-01-0) in order to reach a pH value close to 6 and precipitated in ethanol at 97% (Sigma Aldrich—CAS no. 64-17-5).

The precipitate was solubilized in osmosis water and dialyzed for 48 hours in a dialysis membrane. The solution is then concentrated in Amicon® cells (Millipore) and lyophilized.

### Cell Proliferation Test

This test aims to quantify the impact of functionalization of hyaluronic acid with respect to the proliferation of human dermal fibroblasts. Cell proliferation was evaluated by an ELISA test, based on the detection of the incorporation of bromodeoxyuridine (BrdU ELISA kit—Roche). BrdU is an analog of the nitrogenous bases of the DNA that will be incorporated in it during DNA replication during cell division. It is then possible to detect it with a specific antibody coupled to an enzyme, which will then enable a colorimetric test to be performed and proliferation to be evaluated.

With respect to their precursor, functionalized hyaluronic acids have numerous behaviors depending on their degree of functionalization and their concentration (cf. FIG. 6+table 2 below):
no improvement in proliferation was observed, all concentrations combined: (HH015, HH016, HH017, HH023 and HH024);
the same level of improvement in proliferation was observed, but at optimal concentrations below that of AH 1200: HH020 and HH025 (peak at 0.1 or 0.25 mg/ml compared with 0.5 or 1 mg/ml for AH 1200);
a very clear improvement in proliferation was observed: HH021 and HH022.

TABLE 2
Chemical characterizations of functionalized hyaluronic acids

| | | Total phosphates |
|---|---|---|
| | | (after hydrolysis) |
| HH15 | 0.10 | 0.43 |
| HH16 | 0.10 | 0.44 |
| HH17 | 0.24 | 1.05 |
| HH20 | 0.02 | 0.08 |
| HH21 | 0.07 | 0.30 |
| HH22 | 0.15 | 0.60 |
| HH23 | 0.10 | 0.43 |
| HH24 | 0.10 | 0.42 |
| HH25 | 0.10 | 0.44 |

### Example 3

### Preparation of a Co-Cross-Linked Hydrogel Using a Functionalized Hyaluronic Acid (HH) and Dextran (Process According to the Invention)

### i) Activation of the Hydroxyl Groups of Dextran by a 0.5 M NaOH Solution

0.9 grams of dextran T100 (CAS no. 9004-54-0; Mw=100,000 g/mol) were placed in solution in 50 ml of water, then 5 ml of 0.5 M sodium hydroxide were added to the solution. The mixture was kept under agitation for 30 minutes at room temperature.

### ii) Addition of Functionalized Hyaluronic Acid (HH 22) and Cross-Linking Agent

1.02 grams of sodium trimetaphosphate and 0.2 grams of functionalized hyaluronic acid (HH 22) were added simultaneously (in powder form, spatially separated) to the solution of step a). The mixture was kept under agitation at room temperature until gelling, then the gel obtained was kept for 5 hours at 25° C.

iii) Washing, Purification and Drying of the Hydrogel

The hydrogel was ground on a stainless steel sieve and collected in an acetone solution.

A plurality of washings in water were performed while monitoring the pH, until a pH<7 of the washing water was obtained. For each washing step performed, the hydrogel was filtered on a Büchner with a cellulose acetate filter.

A final washing with acetone was performed, then the hydrogel was dried in the oven under vacuum at 60° C.

### iv) Cytotoxicity

To evaluate the capacity of the cells to adhere and proliferate in the presence of hydrogels based on dextran and functionalized hyaluronic acid, the absence of cytotoxicity of the hydrogels according to the invention was tested. For this, a release of compounds in the culture medium was evaluated. The dry hydrogels were placed in 6-well culture dishes (6 wells per hydrogel) and were left to swell in 4 ml of culture medium:
DMEM (DMEM=Dulbecco's Modified Eagle's Medium—PAA), a cell culture medium that contains amino acids, mineral salts (KCl, MgSO4, NaCl, NaH2PO3), glucose and vitamins (folic acid, nicotinamide, riboflavin and vitamin B12))+2.5% FBS (fetal bovine serum—PAA) for three of the wells;
DMEM+10% SVF for 3 others.

The gels were then left for 72 hours in the incubator at 37° C. The culture supernatants were then collected and centrifuged at 1200 rpm for 5 minutes in order to remove any gel fragment from the supernatants. 200 μl of each supernatant were then added to 96-well culture dishes inoculated 4 hours earlier with 5000 adult human dermal fibroblasts (HDFa—Tebu Bio), in order for the HDFa to have the time to adhere. Each supernatant was tested on three independent wells. The cells were placed in an incubator at 37° C. for 96 hours, then an MTT viability test was performed.

This test is based on the degradation by the mitochondrial dehydrogenases of the tetrazolium salt into formazan, an insoluble violet compound that accumulates in the mitochondria. After incubation for 4 hours at 37° C. of the cells in the presence of 0.625 μg/ml of MTT, the culture medium was removed by pouring and the crystals were dissolved by adding 100 μl of a volume-to-volume mixture of ethanol/DMSO. The reading of the results was then performed by spectrophotometry, at a wavelength of 570 nm.

None of the hydrogels based on dextran and functionalized hyaluronic acid have a cytotoxic effect on human dermal fibroblasts.

### vi) Cell Proliferation Test

Two experimental conditions were tested: (i) inoculation of cells on a thin hydrogel layer; (ii) mixing of the cells in the gel at the time of swelling directly in the culture medium.

Cell viability was evaluated after 72 hours of culture of the cells in the presence of hydrogels. After inoculation of the cells, the hydrogels were digested by eukaryotic hyaluronidase enzymes. A trypan blue exclusion test of viability was performed on the cells collected. The colonization of the hydrogels by the stem cells was observed by optical microscopy and scanning microscopy in order to verify the distribution of cells in the hydrogel.

### Example 4

### Fibroblast Growth Factor (FGF) Release Test

The purpose of this example is to demonstrate the capacity of the hydrogels according to the invention to diffuse growth factors in a controlled manner as they degrade. In this example, four test phosphorylated hydrogels according to the invention (DPD1, DPD2, DPD3, DPD4) and two control hydrogels (DP1, DP2) were synthesized. The control hydrogels DP1 and DP2 are formed only of dextran. The composition of these hydrogels is presented in table 3 below. The compositions may include dextran T100 (100,000 g/mol) and/or dextran T500 (500,000 g/mol).

TABLE 3
Composition of synthesized hydrogels

| | | | % mass | Phosphate | Ratio |
|---|---|---|---|---|---|
| | | % mass | HA or | content | TMP/ |
| Reference | Composition | Dextran | HH | (meq/g) | OH |
| DP1 | Dextran T100 | T100: 95 | 0 | 0.4 | 1.5 |
| (Control) | Dextran T500 | T500: 5 | | | |
| DP2 | Dextran T100 | T100: 90 | 0 | 0.5 | 1.5 |
| (Control) | Dextran T500 | T500: 10 | | | |
| DPD1 | Dextran T100 | T100: 90 | 10 | 0.45 | 1.5 |
| | HA (1200 kDa) | | | | |
| DPD2 | Dextran T100 | T100: 90 | 10 | 0.47 | 1.5 |
| | HH (1200 kDa: | | | | |
| | 0.1 meq/g) | | | | |
| DPD3 | Dextran T100 | T100: 95 | 5 | 0.39 | 1.5 |
| | HH (800 kDa: | | | | |
| | 0.09 meq/g) | | | | |
| DPD4 | Dextran T100 | T100: 85 | 15 | 0.6 | 1.5 |
| | HH (300 kDa: | | | | |
| | 0.1 meq/g) | | | | |

To verify the affinity between the hydrogels according to the invention and the fibroblast growth factors, growth factor release experiments were performed. The synthesized hydrogels (5 mg) were placed in contact with the growth factor (50 ng) at the time of swelling of the gels, then are placed in a saline phosphate buffer solution (SPB) at a temperature of 4° C. After 12 hours of interaction, the buffer solution is replaced by an aqueous sodium chloride solution NaCl 1 M for 12 hours, then by a 1.5 M NaCl solution for 12 hours. Finally, the supernatant fluid is collected in order to determine by assay the percentage of growth factor not absorbed by the hydrogel.

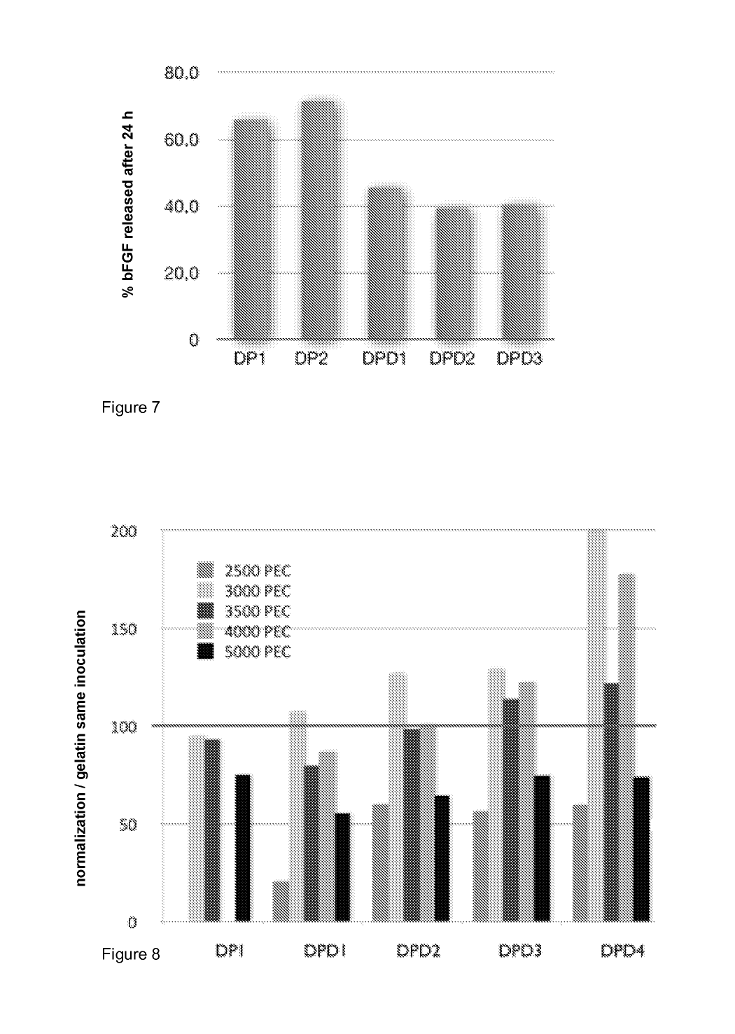

Without being bound to any particular theory, the use of the saline sodium chloride solution, by increasing the ionic force, enables the fibroblast growth factors not absorbed to be specifically separated from the hydrogels tested. According to the hydrogel composition, the absorption capacity of the fibroblast growth factors is different. As shown in FIG. 7, the hydrogels according to the invention (DPD1, DPD2 and DPD3) retain 40% by mass of growth factor after 24 hours of contact in an NaCl solution, by contrast with the control hydrogels (DP1 and DP2) consisting only of dextran, releasing more than 70% growth factors after 24 hours.

The hydrogels according to the invention may therefore be used for a controlled release of growth factor. Thus, a controlled diffusion is possible over time, enabling the growth factors to be released as the hydrogel degrades.

### Example 5

### Cell Proliferation Test

The purpose of this test is to test cell proliferation in the phosphorylated hydrogels according to the invention, in particular on stem cells, and more specifically on circulating endothelial progenitor (CEP) cells. The circulating endothelial progenitors are immature cells, present in the peripheral blood, where their physiological role is to maintain vascular integrity. They originate in the bone marrow, then pass into the blood, where they form a population of very rare cells. They are integrated in the formation of new vessels, for example during the revascularization process following a vascular lesion. In culture, these cells form endothelial cell colonies, which quickly acquire a strong proliferation power. The culture of circulating endothelial progenitor cells is performed on a hydrogel medium, which will enable their survival and proliferation, and, conventionally, gelatin is used as a culture control medium.

In these examples, different initial quantities of CEP stem cells were tested and after 48 hours of culture, a counter of the CEP cells present was performed. The results are expressed as a percentage by mass and are normalized with respect to the total mass of the hydrogel. According to the results, and as shown in the table in FIG. 8, the control hydrogels DP1 and DP2 do not promote or even enable cell proliferation, regardless of the number of cells initially introduced. The hydrogels according to the invention, and more specifically the hydrogel DPD4, is a culture medium clearly promoting cell proliferation since the initial introduction of 3000 cells enables the cell proliferation to be increased by 100% with respect to the hydrogel (cf. result in FIG. 8).

1-12. (canceled)

13. A co-cross-linked phosphorylated polysaccharide hydrogel, comprising:
dextran; and
hyaluronic acid and/or one of its salts, optionally functionalized, wherein said dextran and said hyaluronic acid and/or its salt, optionally functionalized, are bound together by phosphodiester and/or polyphosphodiester covalent bonds.

14. The co-cross-linked phosphorylated polysaccharide hydrogel of claim 13, wherein the hyaluronic acid or its salt is a hyaluronic acid and/or its salt functionalized by sodium trimetaphosphate.

15. The co-cross-linked phosphorylated polysaccharide hydrogel of claim 14, wherein the phosphate concentration of the hyaluronic acid and/or its salt functionalized by sodium trimetaphosphate is between 0.1 and 2 mEq/g.

16. The co-cross-linked phosphorylated polysaccharide hydrogel of claim 13, wherein the phosphate concentration of said hydrogel is between 0.1 and 2 mEq/g.

17. The co-cross-linked phosphorylated polysaccharide hydrogel of claim 13, wherein the co-cross-linked phosphorylated polysaccharide hydrogel is used to prepare a drug to be implanted alone.

18. The co-cross-linked phosphorylated polysaccharide hydrogel of claim 17, wherein the drug is associated with cells for cell therapy.

19. The co-cross-linked phosphorylated polysaccharide hydrogel of claim 17, wherein the drug is in wet form for topical application.

20. The co-cross-linked phosphorylated polysaccharide hydrogel of claim 17, wherein the drug is to deliver active molecules and active principles.

21. The co-cross-linked phosphorylated polysaccharide hydrogel of claim 20, wherein the active molecules and active principles are chosen from:
fibroblast growth factors (FGFs); and/or
platelet derived growth factors (PDGFs); and/or
transformation growth factors (TGFs); and/or
vascular endothelium growth factors (vEGF); and/or
osteoinductive growth factors (BMPs).

22. The co-cross-linked phosphorylated polysaccharide hydrogel of claim 17, wherein the drug is to be used in regenerative medicine.

23. The co-cross-linked phosphorylated polysaccharide hydrogel of claim 17, wherein the drug is for use in human and/or veterinary regenerative medicine.

24. A process for preparing a co-cross-linked phosphorylated polysaccharide hydrogel, based on dextran and hyaluronic acid and/or one of its salts, optionally functionalized, the process comprising:
a) providing a dextran solution;
b) activating at least one hydroxyl group of the dextran by adding an alkaline hydroxide solution to said dextran solution to obtain an activated dextran solution, wherein the concentration of alkaline hydroxide added is between 0.1 M and 0.5 M;
c) adding sodium trimetaphosphate to said activated dextran solution; and
d) adding hyaluronic acid and/or one of its salts, optionally functionalized, to a solution obtained in step c),
wherein said steps a) through d) are performed at a temperature of between 18 and 25° C.

25. The process of claim 24, wherein steps c) and d) are performed concomitantly.

26. The process of claim 25, wherein the sodium trimetaphosphate and hyaluronic acid and/or one of its salts, optionally functionalized, is added concomitantly to the solution obtained in step b).

27. The process of claim 24, wherein the hyaluronic acid and/or one of its salts, optionally functionalized, added in step d) is a hyaluronic acid functionalized by sodium trimetaphosphate.

28. The process of claim 24, wherein said hyaluronic acid and/or one of its salts, optionally functionalized, is added in the form of a powder.

29. The process of claim 24, wherein the alkaline hydroxide solution comprises sodium hydroxide.

30. Use of the hydrogel claim 13 for osteoconductivity and/or osteoinductivity.
